(19) United States
(12) Patent Application Publication
(10) Pub. No.: US 2011/0253156 A1
(43) Pub. Date: Oct. 20, 2011

(54) METHOD FOR SCULPTING EYELASHES

(76) Inventors: Jessica Rose Harley, Chicago, IL (US); Gina C. Mondragon, Chicago, IL (US)

(21) Appl. No.: 12/760,788

(22) Filed: Apr. 15, 2010

(51) Int. Cl. A45D 40/26; A61Q 1/10; A61K 8/87

(52) U.S. Cl. 132/200; 132/218; 424/70.7

(57) ABSTRACT

The present disclosure generally relates to a method for treating eyelashes. The method may include applying the bonding agent directly, via one or more applicators, to the one or more eyelashes and sculpting the bonding agent along a length of the one or more eyelashes to coat and shape the one or more eyelashes. Other embodiments and implementations are also within the scope of the present disclosure.

### TECHNICAL FIELD

This disclosure relates to cosmetics and, more particularly, to a method for sculpting eyelashes using a bonding agent.

### BACKGROUND

Mascaras are well-known in the art and are a major product of the cosmetic industry. Mascaras may be used to enhance the beauty of a person's eyes by coating the eyelashes to thicken, lengthen, color, curl, and define the individual lashes.

Mascaras may come in a variety of forms including cakes, creams, gels, semi-solids, and low viscosity liquids. Some mascaras and other cosmetic compositions have been developed for longer wear and transfer resistance properties. This is typically accomplished by the use of ingredients that form a film after application. However, such compositions generally contain volatile solvents, which may evaporate on contact with the skin or eyelashes, leaving behind a layer comprising waxes and/or resins, pigments, fillers, and actives. These compositions may be uncomfortable for the wearer as the composition remains on the skin or eyelashes as a brittle or non-flexible film. Such compositions may be rigid and hard, and they may not be comfortable to wear. There may also be a tendency for such compositions to flake off because of poor adherence to the skin or eyelashes. Furthermore, such compositions have a tendency to be tacky, resulting in poor application, spreadability and wear characteristics.

A related problem involves the temporary nature of mascara, which requires the wearer to re-apply the mascara daily, often multiple times per day. This often infuriates the wearer as the continual application of mascara becomes both tedious and expensive. Further, current methods for applying mascaras may result in injury to the wearer as the volatile solvents mentioned above may come in to contact with the wearer's eyes or skin.

Another known technique used to improve the look of one's eyelashes involves the attachment of a synthetic eyelash using an adhesive. The adhesive is a semi-permanent black adhesive used to attach a synthetic eyelash extension to a natural lash. This procedure involves dipping a synthetic lash in the adhesive and attaching the synthetic lash to the person's natural eyelash.

### SUMMARY OF DISCLOSURE

In an embodiment of the present disclosure a method for sculpting eyelashes is provided. The method may include providing a bonding agent and applying the bonding agent directly, via one or more applicators, to the one or more eyelashes. The method may further include sculpting the bonding agent along a length of the one or more eyelashes to coat and shape the one or more eyelashes.

One or more of the following features may be included. The bonding agent may include cyanoacrylate ester. The bonding agent may also include one or more of Ethyl Cyano Acrylate, Poly Alkyl Methacrylate, Polyisocyanate, Monomer, and Pigment. The bonding agent may also be partially or entirely waterproof.

In some embodiments, applying the bonding agent may include applying the bonding agent to at least one of an upper set and a lower set of the one or more eyelashes. The method may further include applying a patch to a lower set of the one or more eyelashes. Applying may be performed using one or more of a comb applicator, a mini applicator, a precision swab and a precision brush applicator. The method may further include applying one or more of a toner, a refining solution, and a sealant.

In another embodiment of the present disclosure, a kit for treating eyelashes is provided. The kit may include a bonding agent, one or more applicators configured to apply the bonding agent and a housing configured to contain the bonding agent and the one or more applicators.

One or more of the following features may be associated with the eyelash kit. The bonding agent may include cyanoacrylate ester. The bonding agent may also include one or more of Ethyl Cyano Acrylate, Poly Alkyl Methacrylate, Polyisocyanate, Monomer, and Pigment. The bonding agent may also be partially or entirely waterproof.

In some embodiments, the kit may include one or more applicators including, but not limited to, a comb applicator, a mini applicator, a precision swab and a precision brush applicator. The kit may further include one or more of a toner and a refining solution.

In another embodiment of the present disclosure an eyelash enhancement compound is provided. The eyelash enhancement compound may be configured to enhance the appearance of one or more eyelashes. In some embodiments, the compound may include one or more of Ethyl Cyano Acrylate, Poly Alkyl Methacrylate, Polyisocyanate, Monomer, and Pigment. Other ingredients are also within the scope of the present disclosure.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is diagram showing a set of eyelashes prior to receiving the method of the present disclosure;

FIG. 2 is a diagram showing the set of eyelashes with surgical tape applied under the lower set of eyelashes;

FIG. 3 is a diagram showing the set of eyelashes with a patch applied atop lower set of eyelashes;

FIG. 4 is a diagram showing the set of eyelashes after the method of the present disclosure has been performed;

FIG. 5 is a diagram showing a precision swab used in the method of the present disclosure;

FIG. 6 is a diagram showing a comb applicator used in the method of the present disclosure;

FIG. 7 is a diagram showing a tapered and straight mini applicators used in the method of the present disclosure;

FIG. 8 is a diagram showing a precision brush applicator used in the method of the present disclosure;

FIG. 9 is a flowchart depicting operations consistent with the method of the present disclosure; and

FIG. 10 is an embodiment of an eyelash treatment kit consistent with the methods of the present disclosure.

### DETAILED DESCRIPTION OF THE PREFERRED EMBODIMENTS

Generally, the present disclosure is directed towards a method for sculpting eyelashes. In some embodiments, the teachings of the present disclosure may be used to replace the need for traditional mascara by providing a more permanent treatment.

As described in further detail hereinbelow, a bonding agent may be applied to one or more eyelashes. Once applied, a finish similar to that of a mascara application is produced, however, a more permanent result is obtained. In this way, the present disclosure may be used to coat and/or color each lash with a deep finish, which may approximately correspond with the person's natural eyelash color. A variety of different colors may be employed, including but not limited to, blue, green, yellow, purple, white, black, etc. For example, the color may range tonally depending upon the desired finish, i.e., black noir to blackest black. It may also be clear in color.

The term “bonding agent” as used herein is meant to refer to a substance capable of adhering to one or more eyelashes. The bonding agent may have a color approximately corresponding to the color of a client's eyelashes. In some embodiments, the bonding agent may include cyanoacrylate ester and/or a polymeric substance such as an elastomer. In some particular embodiments the elastomer may include urethane rubber. In some embodiments, the bonding agent may include one or more of the following: Ethyl Cyano Acrylate, Poly Alkyl Methacrylate, Polyisocyanate, Monomer, and/or Pigment.

The bonding agent may provide a number of functions, including, but not limited to, curving, holding, and setting lashes, as well as in separating, increasing lash volume and extending lash length. In some embodiments, the bonding agent used to coat the lashes may be waterproof and may create a dark finish on lashes. The bonding agent may be an FDA approved, medical grade solution approved for use near the eye.

In some embodiments, the bonding agent may be similar to a semi-permanent black adhesive used to attach a synthetic eyelash extension to a natural lash. However, the present disclosure describes methods in which the entire eyelash is coated with this adhesive creating a semi-permanent finish. The present disclosure may include applying the bonding agent directly to the eyelashes using various applicators and sculpting the bonding agent along a length of the one or more eyelashes to coat and/or shape the lashes.

While the appearance of the finish may be similar to that of mascara, the long-lasting effects provided by the present disclosure improve upon the temporary aspect of traditional mascara. In other words, it is the application of this bonding agent directly to the eyelash itself, without attaching a fake eyelash that is proposed. This results in a cosmetic enhancement method, with a lasting result that does not currently exist in the art.

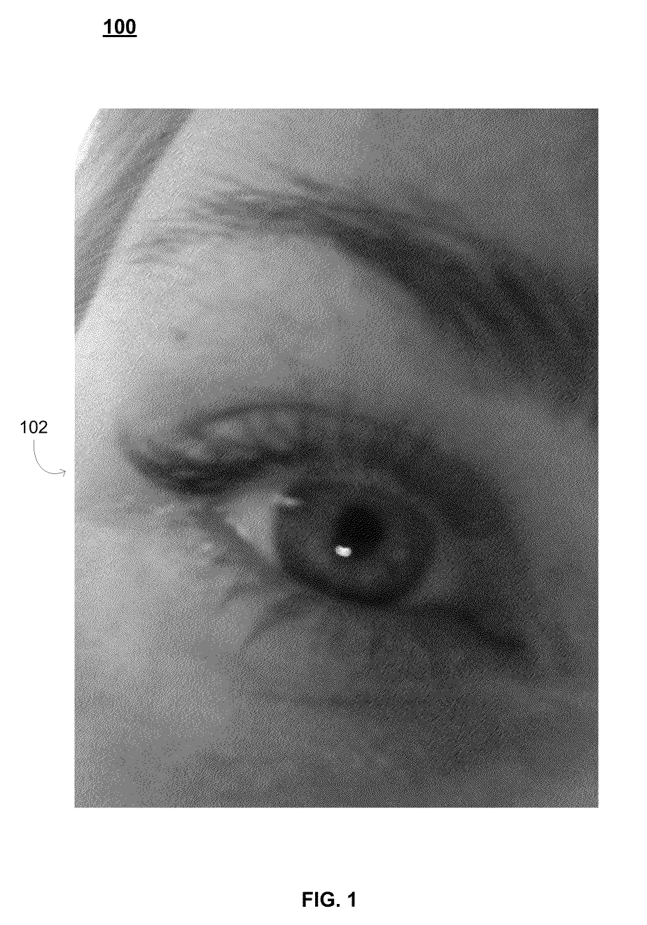

Referring now to FIG. 1, a diagram 100 is provided showing untreated eyelash set 102. The method of the present disclosure may be comfortably performed while the client lies down. In some embodiments, the initial application may require approximately 1 hour, while touch ups may require approximately 30 minutes. These timeframes are provided merely for exemplary purposes as other timeframes are also possible.

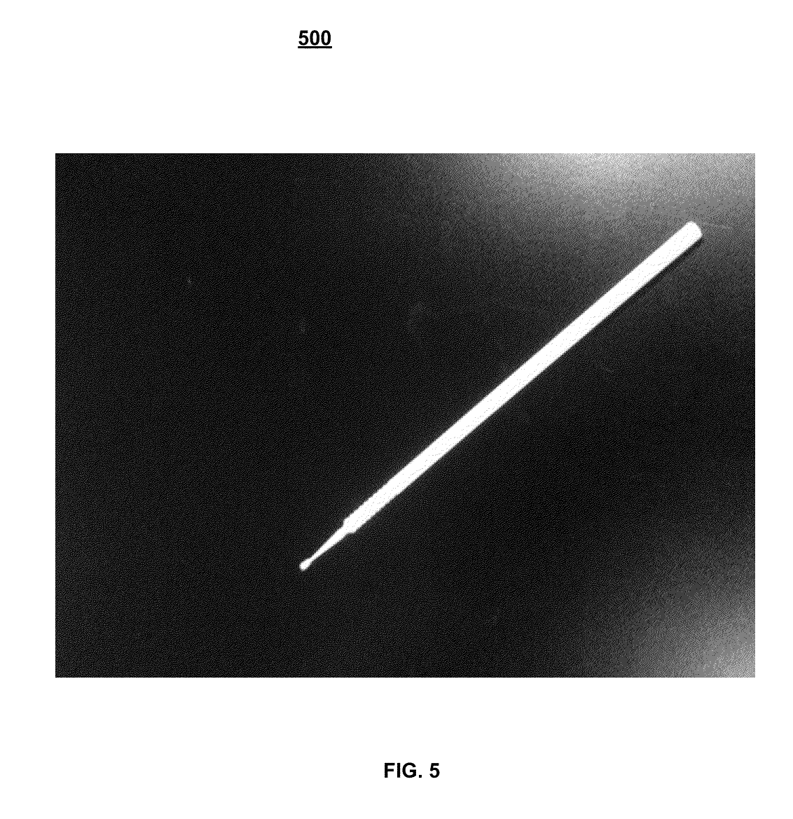

In operation, and for the majority of the procedure, a client's eyes may remain closed. In some embodiments, the upper lashes may be “dipped” first, then the lower lashes, using a number of application tools, which are described in further detail below and are shown in FIGS. 5-8. The bonding agent may be applied along the entire length, or substantially along the entire length of the client's eyelashes.

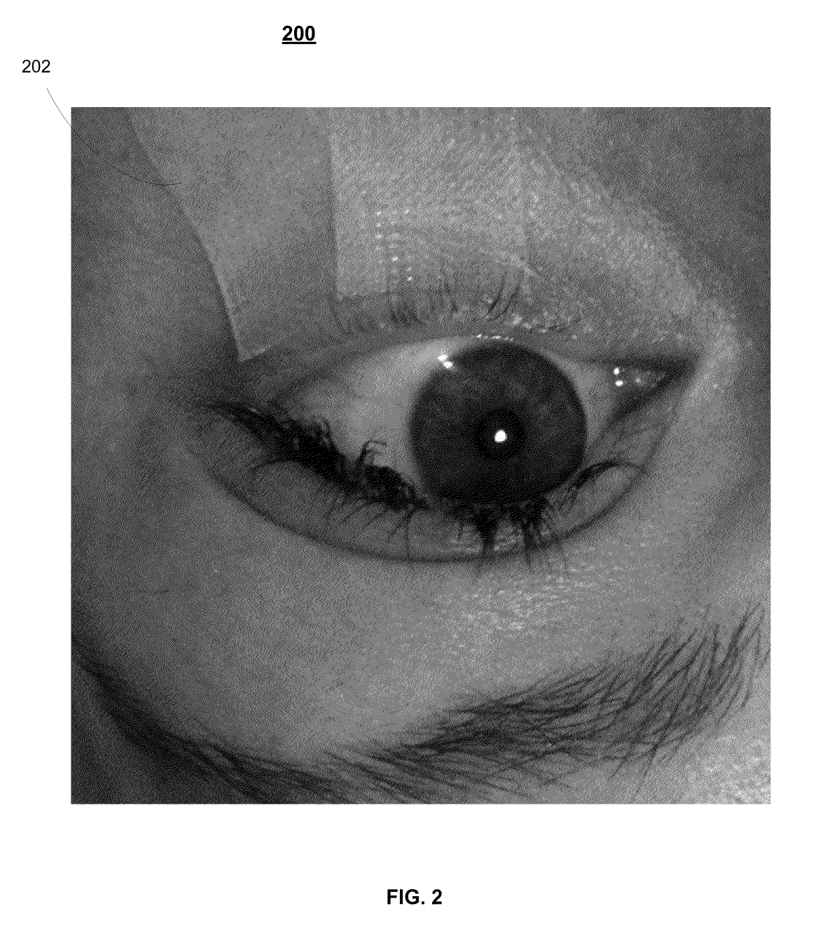

Referring now to FIG. 2, an embodiment 200 depicting operations associated with the methods of the present disclosure is provided. In this embodiment, an adhesive 202, including but not limited to, surgical tape, may be used to separate the skin during application. The tape may be used to protect the skin from contact with the lower lashes during application. Adhesive 202 may also be configured to protect the client's lower set of eyelashes and to keep the eyepad or patch in place, which is discussed in further detail below. FIG. 2 depicts adhesive 202 as applied to the skin beneath the lower set of eyelashes in order to protect the lower lashes during upper lash application, and protect the skin during lower lash application.

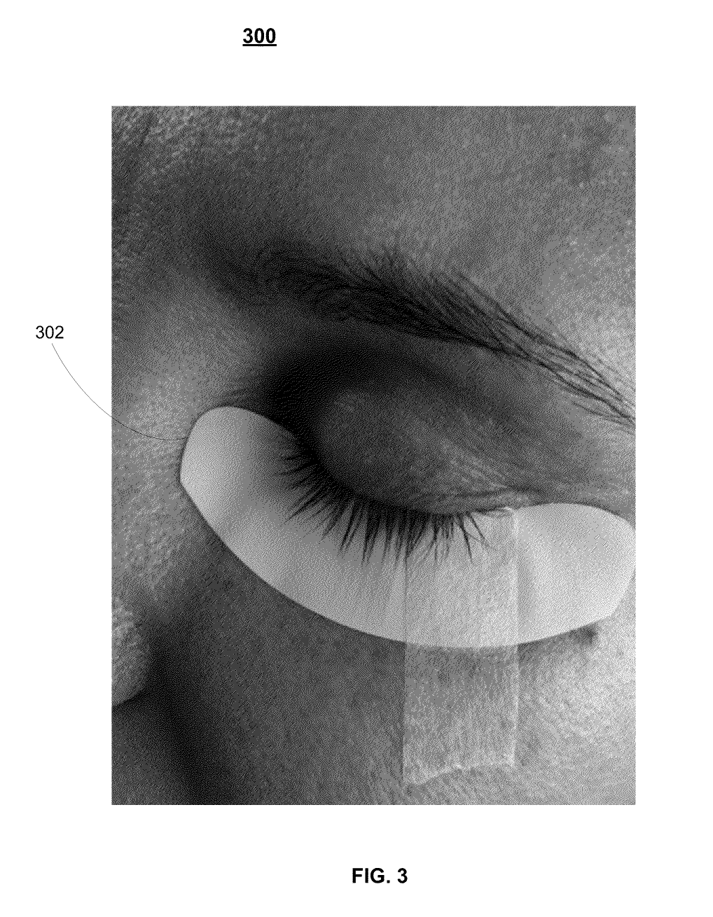

Referring now to FIG. 3, an embodiment 300 depicting operations associated with the methods of the present disclosure is provided. A patch 302 may be placed over the top of the lower lashes to cover and protect them during the upper lash application procedure. Patch 302 may be configured to prevent the upper & lower lashes from bonding together. In some embodiments, patch 302 may be an eyepad or similar skin protectant, which, in addition to protecting the set of lower eyelashes, may also be configured to protect the client's under-eye skin during the application of the bonding agent to the set of upper eyelashes. Patch 302 may also provide a moisturizing under-eye treatment to the client during application of the bonding agent.

In some embodiments, a number of additional solutions may be applied to the eyelashes in addition to the bonding agent. For example, a toner may be applied prior to the bonding agent. The toner may be a liquid solution that may be applied using a disposable or non-disposable applicator. The toner may be used to clean and prepare the lashes to be treated by removing any natural oils produced by the body, and/or any debris from the lashes. Once the toner has been applied the eyelashes may be ready to receive the bonding agent. A corrector may be applied after the bonding agent. The corrector may be a liquid dissolving agent configured to reverse undesired results or mistakes made during the application process. This corrector may also be capable of dissolving the bonding agent's adherence to the lash. Following this, a sealant may be applied to the lashes using a disposable or non-disposable applicator. This may assist in fortifying the hold of the bonding agent to the lashes and may assist in sustaining the desired finish.

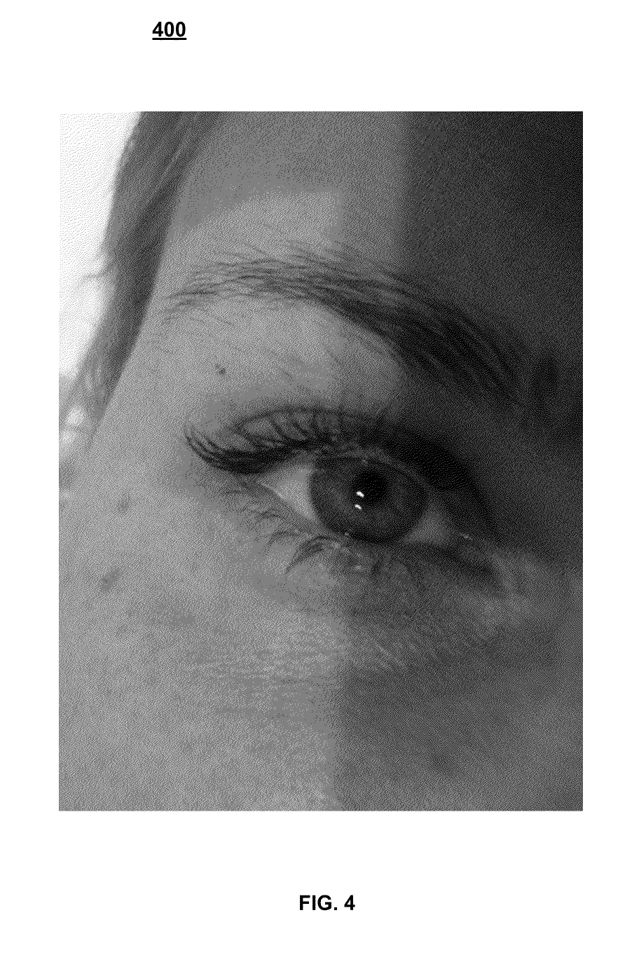

In some embodiments, multiple coats of the bonding agent may be applied to the eyelashes depending on the level of lash enhancement desired. Each individually applied coat may be properly dried before the next should be applied. Once the desired enhancement is achieved and properly dried the client may open their eyes without any discomfort. The bonding agent may take approximately 10 seconds to dry. Complete curing of the bonding agent may be dependent on how many coats are applied. Normal activity may be resumed immediately after the initial application, however, it may be best to treat the lashes post-application with prescribed care for the first 2-12 hours. FIG. 4 depicts an embodiment 400 showing a set of eyelashes after the methods of the present disclosure have been performed.

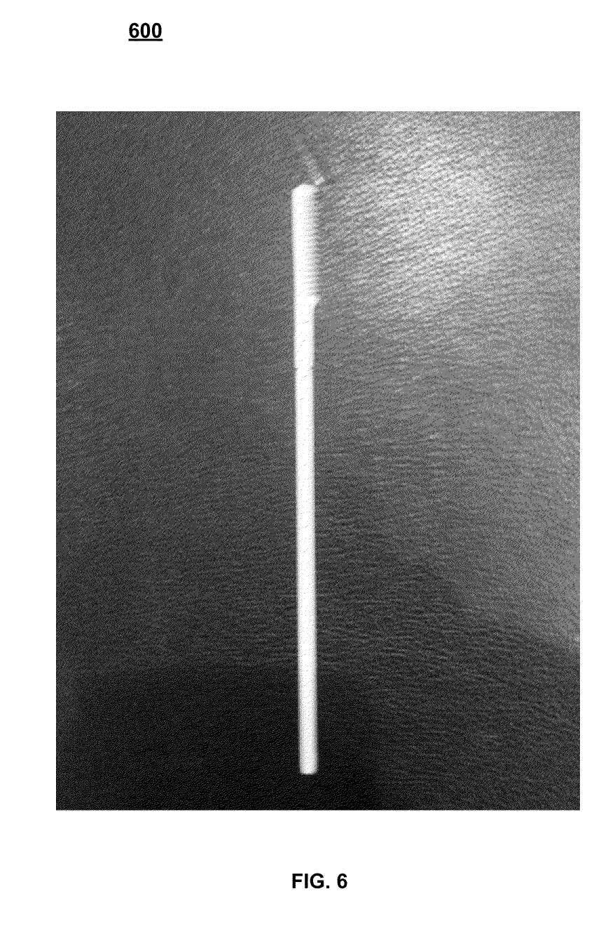

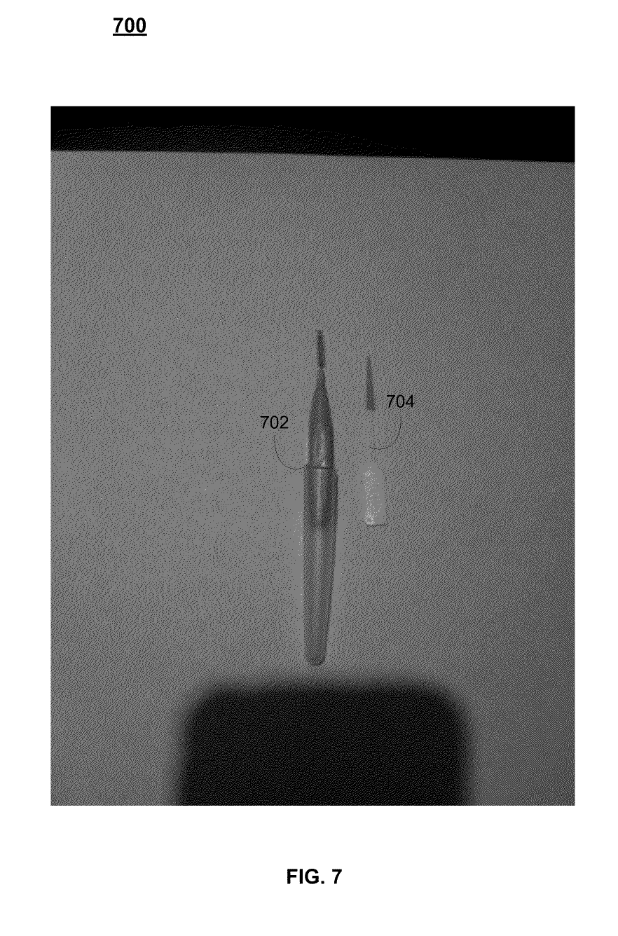

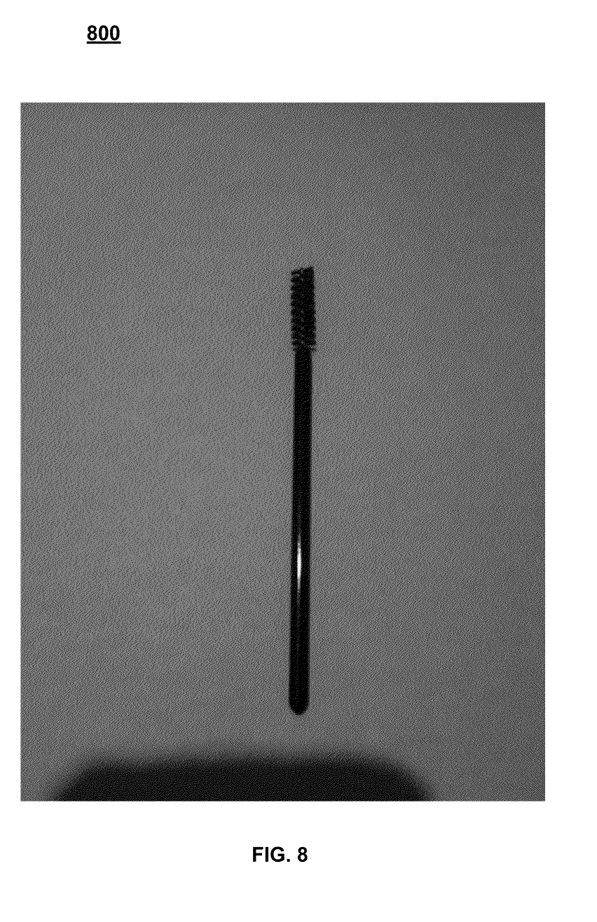

Referring now to FIGS. 5-8, embodiments showing some of the applicators that may be used to sculpt, coat and/or shape various solutions, e.g. the bonding agent, to the eyelashes. FIG. 5 depicts and embodiment 500 of a precision swab, which may be used to clean the eyelashes both before and after application of the bonding agent. The precision swab may also be used to apply the bonding agent, toner, refining solution, and sealant to the eyelashes. FIG. 6 depicts an embodiment 600 of a comb applicator, which may be used to apply the bonding agent to the upper and lower sets of eyelashes. FIG. 7 depicts an embodiment 700 of tapered 702 and straight 704 precision applicators configured to apply the bonding agent to the upper or lower sets of eyelashes. FIG. 8 depicts an embodiment 800 of a brush applicator, which may be used to brush through and separate the eyelashes prior to application of the bonding agent.

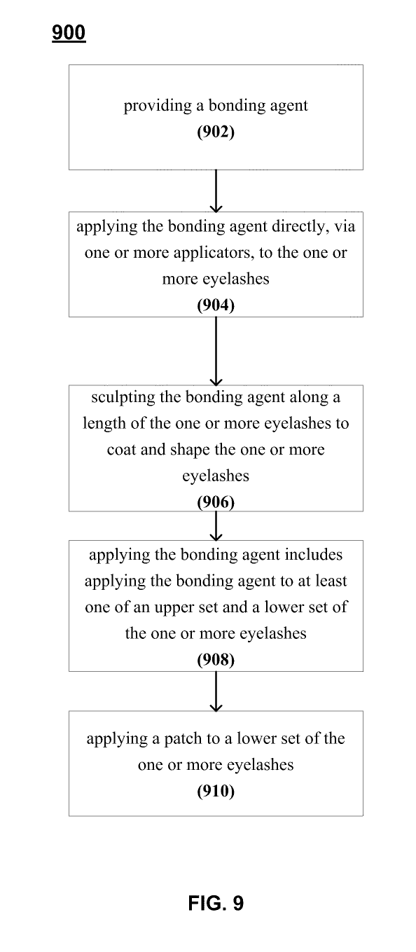

Referring now to FIG. 9, an embodiment 900 depicting operations consistent with the method of the present disclosure is provided. The method may include providing a bonding agent having a color approximately corresponding to a color of one or more eyelashes (902). The method may further include applying the bonding agent directly, via one or more applicators, to the one or more eyelashes (904) and sculpting the bonding agent along a length of the one or more eyelashes to coat and shape the one or more eyelashes (906). The method may also include applying the bonding agent to at least one of an upper set and a lower set of the one or more eyelashes (908). The method may further include applying a patch to a lower set of the one or more eyelashes (910). In some embodiments, applying the patch may be the first operation of the method.

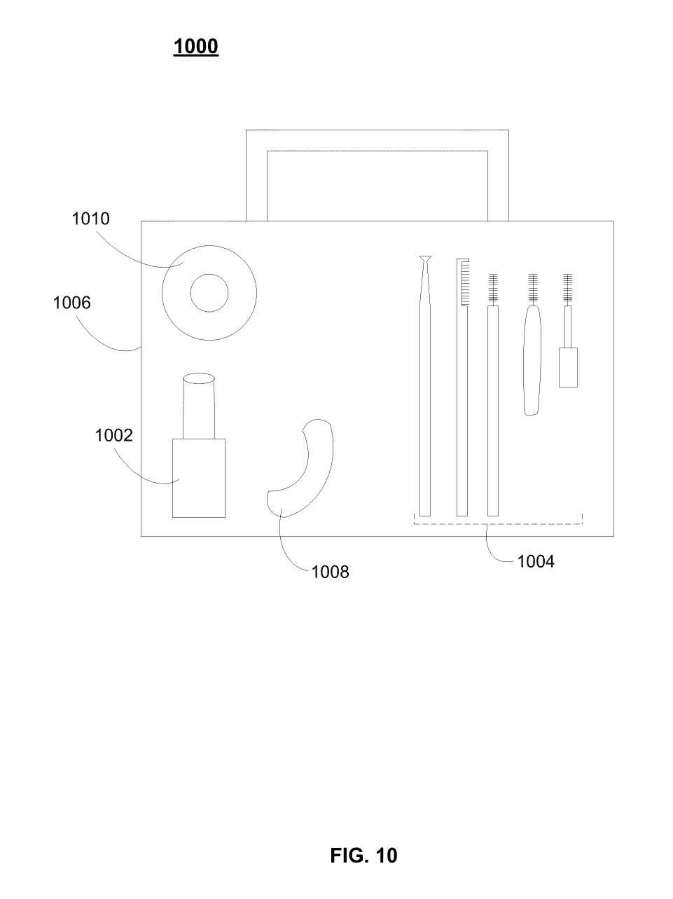

Referring now to FIG. 10, an eyelash treatment kit 1000 is provided. Kit 1000 may include any or all of the components described herein. For example, kit 1000 may include a bonding agent 1002 having a color approximately corresponding to a color of one or more eyelashes. Kit 1000 may further include one or more disposable and/or non-disposable applicators 1004 configured to apply bonding agent 1002. Kit 1000 may further include eyepads 1008, tape 1010, and any other of the components described herein. Some of all of the components of kit 1000 may be enclosed within a housing 1006 configured to contain bonding agent 1002 and the one or more disposable and/or non-disposable applicators 1004. Housing 1006 may be a resealable package, which may be transparent in nature and packaged for sale. For example, in one particular embodiment kit 100 may include a straight needle, tweezers-bent, flocked precision applicators (e.g., disposable), white comb applicators (e.g., disposable), brush applicators (e.g., disposable), precision applicators (e.g., disposable), tool prep pads (e.g., disposable), solutions trays (e.g., disposable), eye area protective pads (e.g., disposable), surgical tape, precision swab applicators (e.g., disposable), cotton swabs (e.g., disposable), practice face model, solutions (e.g., 1 bottle of each per kit—listed in order of application): toner (lash prep solution), bonding agent, refining solution, and sealant. An air dryer and magnification instruments may also be supplied.

In some embodiments, the present disclosure may also include an eyelash enhancement compound. The eyelash enhancement compound may be configured to enhance the appearance of one or more eyelashes. The eyelash enhancement compound may be applied directly to an entire length of one or more eyelashes to provide the benefits described above. In some embodiments, the compound may include cyanoacrylate ester and a polymeric substance such as an elastomer (e.g., urethane rubber). In some embodiments, the eyelash enhancement compound may include one or more of Ethyl Cyano Acrylate; Poly Alkyl Methacrylate; Polyisocyanate; Monomer; and Pigment.

In some embodiments, the methods described herein may set forth a procedure that requires in-salon application by a certified professional. Since the bonding agent is applied to the natural lash and not the skin, any contact with the skin surrounding the eye area is avoided. The bonding agent, after proper curing, may create a waterproof bond to each individual eyelash to which it is applied. The finish of bonding agent also maintains its semi-permanency and tone against sweat and/or tears.

The methods of the present disclosure may provide significant advantages over the prior art. Unlike traditional mascara currently available, one does not need to have the bonding agent applied each day. This method may be used to create a semi-permanent finish on the lashes. This finish may last throughout the life cycle of one's natural lash. As our natural lashes grow out, producing “roots”, touch-up application may be necessary about every three to six weeks. Touch-up application may also be necessary due to one's lash life cycle. A typical person sheds their natural lashes approximately every 60 to 100 days. With each natural lash shed, a new lash grows in its place. This is the natural life cycle of an individual's eyelashes, which may vary depending upon the individual. If a touch-up is not desired the client can simply allow the effect of the present method to grow out in the duration of their natural lash life cycle, or have the application removed by a certified professional.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Any order of operations depicted in the flowcharts or in the specification is merely for exemplary purposes, unless stated otherwise.

A number of implementations have been described. Nevertheless, it will be understood that various modifications may be made. Accordingly, other implementations are within the scope of the following claims.

What is claimed is:

1. A method for treating eyelashes comprising:
providing a bonding agent;
applying the bonding agent directly, via one or more applicators, to the one or more eyelashes; and
sculpting the bonding agent along a length of the one or more eyelashes to coat and shape the one or more eyelashes.

2. The method of claim 1 wherein the bonding agent includes cyanoacrylate ester.

3. The method of claim 1 wherein the bonding agent includes an elastomer.

4. The method of claim 1 wherein the bonding agent includes one or more of Ethyl Cyano Acrylate, Poly Alkyl Methacrylate, Polyisocyanate, Monomer, and Pigment.

5. The method of claim 1 wherein the bonding agent is waterproof.

6. The method of claim 1 wherein applying the bonding agent includes applying the bonding agent to at least one of an upper set and a lower set of the one or more eyelashes.

7. The method of claim 6 further comprising applying a patch to a lower set of the one or more eyelashes.

8. The method of claim 1 wherein applying is performed using one or more of a comb applicator, a precision applicator, a precision swab and a brush applicator.

9. The method of claim 1 further comprising applying a toner configured to cleanse the one or more eyelashes.

10. The method of claim 1 further comprising applying a corrector configured to dissolve at least a portion of the bonding agent.

11. The method of claim 1 further comprising applying a sealant to the one or more eyelashes.

12. A kit for treating eyelashes comprising:
a bonding agent;
one or more applicators configured to apply the bonding agent directly to the one or more eyelashes to coat and shape the one or more eyelashes; and
a housing configured to contain the bonding agent and the one or more applicators.

13. The kit of claim 12 wherein the bonding agent includes cyanoacrylate ester.

14. The kit of claim 12 wherein the bonding agent includes an elastomer.

15. The kit of claim 14 wherein the bonding agent includes one or more of Ethyl Cyano Acrylate, Poly Alkyl Methacrylate, Polyisocyanate, Monomer, and Pigment.

16. The kit of claim 12 wherein the bonding agent is waterproof.

17. The kit of claim 12 wherein the one or more applicators includes one or more of a comb applicator, a precision applicator, a precision swab and a brush applicator.

18. The kit of claim 12 further comprising applying a toner configured to cleanse the one or more eyelashes.

19. The kit of claim 12 further comprising applying a corrector configured to dissolve or alter the finish of at least a portion of the bonding agent.

20. An eyelash enhancement compound configured to be applied directly to a length of one or more eyelashes to enhance their appearance, the compound comprising:
Ethyl Cyano Acrylate;
Poly Alkyl Methacrylate;
Polyisocyanate;
Monomer; and
Pigment
